(19) United States
(12) Patent Application Publication
(10) Pub. No.: US 2004/0086472 A1
(43) Pub. Date: May 6, 2004

(54) Method for demonstrating universality of flesh tones

(76) Inventors: Margie L. Porter, Tipton, IN (US); Susan K. McGregor, Cincinnati, OH (US); Orpha Crouch, Saginaw, TX (US)

Correspondence Address: Margie L. Porter, 118 Maple Street, P.O. Box 185, Tipton, IN, 46072, US

(21) Appl. No.: 10/289,466

(22) Filed: Nov. 5, 2002

(51) Int. Cl. A61K 7/021; G06F 17/60

(52) U.S. Cl. 424/63; 705/2

(57) ABSTRACT

A method for blending liquid or powdered paints (11, 13-14) in colors of red, yellow, and blue, in some cases tinted with white or shaded with black (1-10) to produce sample skin tones. Demonstration that the skin of every race is a blend of primary colors with or without tint (white) or shade (black) added. A multicultural practical application is included (15-24). The user blends and applies the paint based on his or her perceptions of what skin colors look like on himself or herself as well as on other persons.

### BACKGROUND

1. Field of Invention

This invention relates to educational and demonstrational aspects of painting and art, specifically to visual art, and the demonstration or training of blending primary colors to produce flesh tones.

2. Description of Prior Art

The teaching of multiculturalism as relates to skin color has been a publicly debated issue at least since 1962 when, during the Civil Rights movement, the Crayola Company decided to rename their “Flesh” crayon and call it “Peach.” Perception of flesh tones was also the stated issue when that same company renamed their “Indian Red” crayon in 1999 and began calling it “Chestnut.” Today, crayons in multiple shades of brown and premixed multicultural paints are available on the market. Flesh colored paint is also used in, for example, U.S. Pat. No. 4,828,116, which is a process for making a representation of a portion of a person's body. These products allow users to depict persons of various racial groups but do not address issues of the perception of color. Nor do they allow the user to discover that flesh tones are comprised of various tints and shades of the primary colors: red, yellow, and blue.

One doll invented by Bellavia and described in patent application Ser. No. 20010046830, Class 446/369 (2001) has interchangeable external characteristics, including skin that can be added to a model with humanoid organs and skeletal features to demonstrate the relationship between inner and outer anatomy. This invention, using manipulation of toy parts, demonstrates that persons of different races are similar, but it does not allow the freedom of expression inherent in painting and art.

Processes for blending pigments to create flesh-toned cosmetics have been invented. U.S. Pat. No. 6,284,228 to Markowitz and Markowitz (2001) allows the user to create a personalized foundation by blending pigments into a white base to create flesh tones. U.S. Pat. No. 6,316,526 to Lugert (2001) uses a heated mixture of crushed materials, fatty acids, and water to make leads for colored pencils, cosmetic pencils, and colored chalk. Pigments are used to create a variety of colors and hues of nail polish in U.S. Pat. No. 5,778,901 to Abrahamian (1998). However, cosmetics and nail polishes are not generally used in artwork.

A process for matching a person's skin tone is offered by Heincke in U.S. Pat. No. 6,297,420 (2001). Heincke's method uses heat or light to activate ingredients which produce a desired skin tone so that the color may be scanned and stored. A color comparison reference is described in U.S. Pat. No. 4,523,852 to Bauer (1983). A device and method for selecting cosmetics is proffered in U.S. Pat. No. 5,178,169 to Lamle (1991). U.S. Pat. No. 5,727,567 uses clear and pigmented layers to construct artificial skin. These inventions demonstrate that human skin is a variety of colors, but they do not allow the user to see the individual red, yellow, and blue components of the skin tones.

A color measurement system for skin, hair, and teeth, which is used to help evaluate medical conditions, is described in U.S. patent application Ser. No. 20020021444 by Macfarlane (2002). The color index included in this invention is used primarily to evaluate color changes which would indicate an increase in bilirubin or indicate a jaundiced condition, but it also has cosmetic applications. Although this invention shows the components of color found in skin, the invention is accessible only to persons who have the technical training to utilize the device.

Color theory and a three-dimensional character of color perception are included in U.S. Pat. No. 5,537,228 to Dillinger (1996). Dillinger's apparatus forms color images with perceptual variables using colored lights to stimulate the retina of the eye. Use of this device also requires formal technical training.

Photochromatic flesh-colored pigments have been invented for example, by Ohno, et al. as described in U.S. Pat. No. 5,176,905 (1993). Our invention, however, refers to manual paints and colors vs. film art.

Correction of a hue is addressed in U.S. Pat. No. 5,513,991 to Reynolds, et al. (1996) in an art instruction compact disk. Methods of blending colors are described in, for example, U.S. Pat. No. 3,815,265 to DePauw (1974), U.S. Pat. Nos. 4,597,997 to Weill (1986), and 4,009,527 to Scott and Zimmerman (1977). These inventions do not specifically address or demonstrate the commonality among skin colors, as our invention does.

### OBJECTS AND ADVANTAGES

The use of high tech equipment or high dollar components is prohibitive for use in a home or teaching situation. Our interdisciplinary method of teaching about multiculturalism by blending measured amounts of paint according to formulas as described in our copyrighted instruction booklet, Color My World: Color My Skin, requires only supplies that are inexpensive and readily available to the average teacher or parent. The needed supplies include:

liquid or dry paint, either pre-measured, or accompanied by any standardized measure, which may include measuring cups and spoons, graduated beakers or tubes, syringes or eyedroppers marked in increments, or household objects, such as bottle caps or film canisters.

paint brushes of any size or quality that may be reasonably manipulated by the user.

clean containers of adequate size to hold the mixed paint.

water to be blended into dry or powdered paint.

a stick or spoon for mixing and stirring the paint.

a painting surface or paper, preferably in a natural tone, as a white surface tends to distort flesh tones. Inexpensive painting pads made of newspaper stock are ideal.

Our teaching method also demonstrates that color is an idea, or a perception. Therefore, grass green is an individual's idea of grass green, and each person may perceive skin color in a different way.

Accordingly, several objects and advantages of our invention become clear. Our invention:

(a) enhances multicultural education in regard to the differences and similarities among skin colors.

(b) provides a method of depicting persons of various races and skin colors using inexpensive powdered, liquid, or washable paints.

(c) demonstrates that color is perception rather than material fact.

(d) illustrates that skin tones of all races are comprised of various tints and shades of the primary colors: red, yellow, and blue.

(e) makes color theory as related to skin tone accessible to students and persons with no explicit formal or technical training.

(f) addresses the commonality among skin colors.

(g) utilizes interdisciplinary methods of mixing flesh colored paints, so that students and other persons who use our invention are enabled to learn about art, social studies, and math simultaneously. In grades 6+, scientific measuring is also included in this activity.

### SUMMARY

The present invention is a method of mixing dry or liquid paints according to formulas, resulting in mixtures that mimic the flesh tones of various races and cultures. The invention includes a painting activity that allows the user to discover his or her own perceptions of skin colors.

### DRAWINGS

FIG. 1-FIG. 10 show formulas, which are adaptable to many units of measure. Depending upon the user's level of fine motor skills, these units may range from milliliters to measuring cups.

FIG. 11 illustrates that the liquid paints may be mixed in an ordinary cup, jar, or other container of adequate size.

FIG. 12 illustrates that any stick or spoon of adequate length to reach from the bottom of the container to the user's hand may be used to mix and stir the paint.

FIGS. 13-14 illustrates the use of powdered paint, which is blended together, then water is added and mixed in to create liquid paint.

FIG. 15-FIG. 24 are sample captioned drawings which illustrate the practical application of our invention, allowing the user to depict people in various walks of life according to the user's own perceptions of skin color.

### DETAILED DESCRIPTION

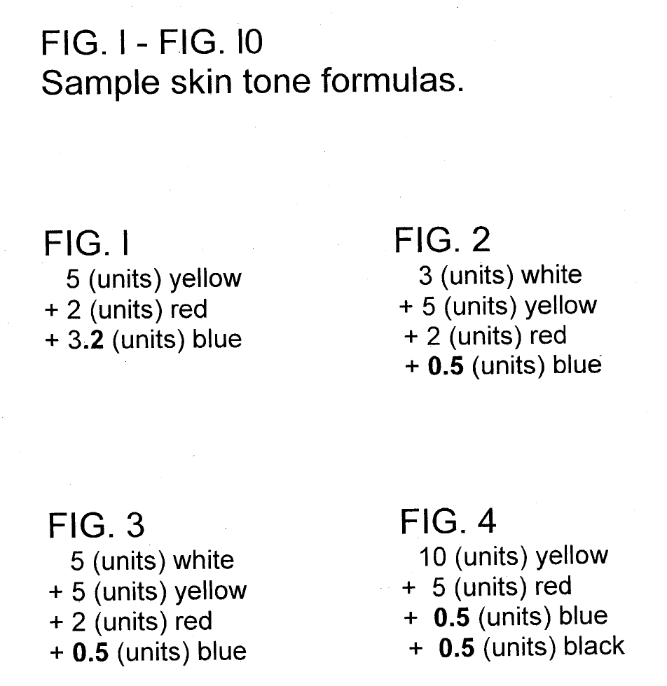

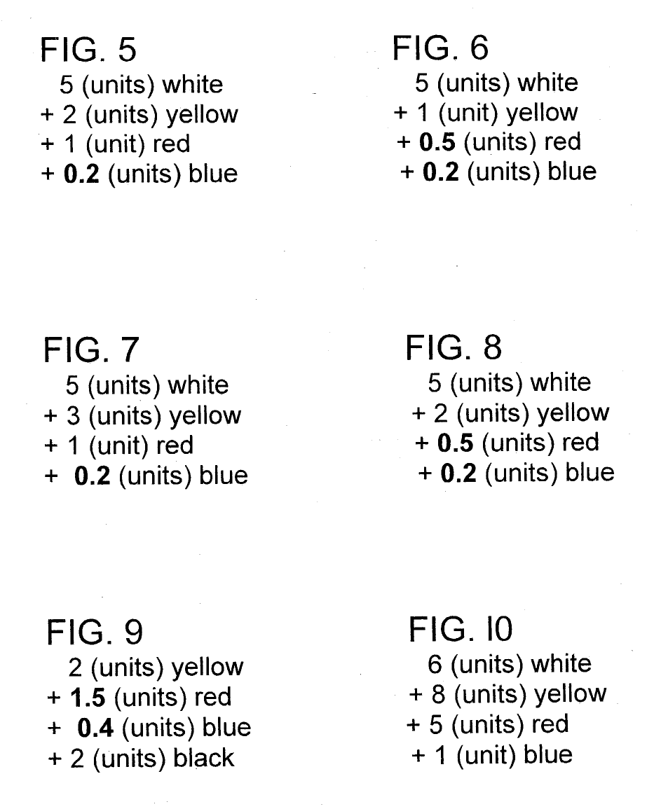

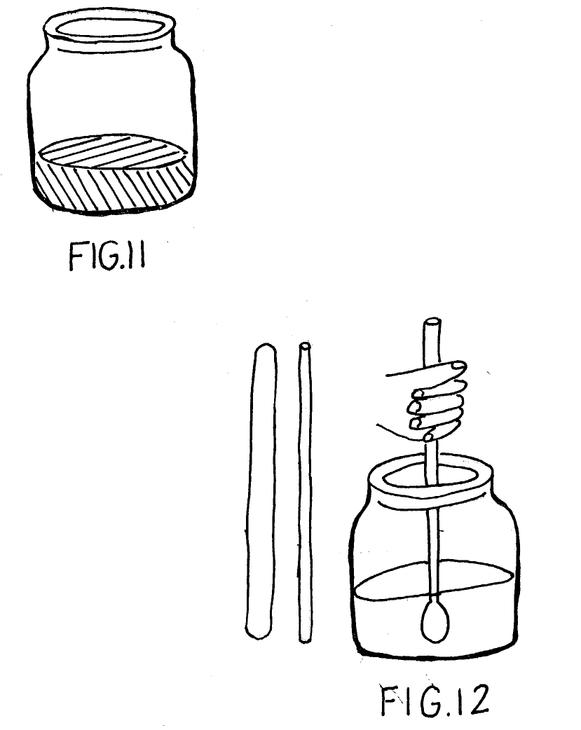

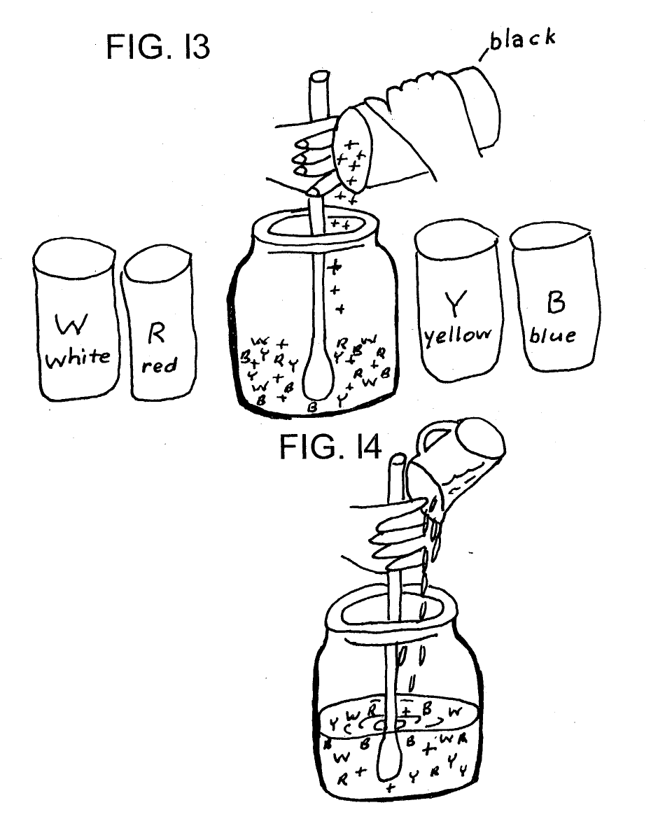

A preferred embodiment of the present invention is illustrated in FIG. 1-FIG. 10. The base formulas were created using Crayola brand washable paint in colors of red, yellow, blue, white, and black. These formulas can be adapted to similar paints produced by the Prang and Rose Art companies. Test surfaces for the blended colors include newspaper stock, manila paper, and children's art books published by Golden Books, Hasbro, Playmore, and Landoll's.

When measured amounts of paint are blended in a clean container (FIG. 11), and mixed with an instrument that simultaneously reaches both the user's hand and the bottom of the container (FIG. 12), the resulting mixture mimics a common flesh tone. Each formula produces a different skin color.

### ADVANTAGES

From the description above, a number of advantages of our method for demonstrating the universality of flesh tones become evident:

(a) Persons without formal art training will be able to use colorants or paint to produce skin colors.

(b) Supplies used in our method are inexpensive, readily available, and often on hand in schools, churches, and youth clubs.

(c) The method illustrates that the difference in the skin colors of individuals may be no more than a degree of red, yellow, or blue.

(d) The method can be adapted to use products supplied by a variety of companies.

### CONCLUSION, RAMIFICATIONS, AND SCOPE OF INVENTION

Thus the reader will see that the color mixing method of our invention provides a highly reliable, yet economical method for demonstrating the universal components of skin colors that can be used by persons of any age who have average motor skills.

While our above description contains many specificities, these should not be construed as limitations on the scope of the invention, but rather as exemplification of one preferred embodiment thereof. Many other variations are possible. For example:

The colors can be used for animals and natural objects as well as skin.

The colors may be applied individually, in tiny dots, using paint, crayon, colored pencils, or other media, rather than blended together.

The proportions may be adapted to other media, such as uniformly and proportionally weighed pieces of crushed chalk or melted wax crayon.

Accordingly, the scope of the invention should be determined not by the embodiment(s) illustrated, but by the appended claims and their legal equivalents.

We claim:

1. A method for demonstrating the commonalities of multiethnic and multiracial skin colors wherein said skin colors are composed from formulated amounts of colorants, said colorants being the primary colors, red, yellow, and blue, plus white tints and black shades of said colorants comprising
(a) the colorant of claim 1, wherein the colorant is a liquid paint.
(b) the colorant of claim 1, wherein the colorant is a powdered dry paint.

2. The formulated units of colorant according to claim 1, wherein the units of liquid or dry colorant are measured in equivalent units of weight or volume to produce skin tones.
(a) the said equivalent units of claim 2, wherein said colorants of various manufactures and qualities may be altered with more or less quantities of the colorants red, yellow, blue, black, and white to produce said multicultural and multiracial skin colors.
(b) the said equivalent units of claim 2, wherein a painting or coloring surface used for said method of claim 1, may be used by altering said formulas with more or less quantities of the colorants red, yellow, blue, black, and white, to produce said multiracial and multicultural skin colors on said surfaces of various textures and hues.
